(19) United States
(12) Patent Application Publication
(10) Pub. No.: US 2023/0277729 A1
(43) Pub. Date: Sep. 7, 2023

(54) Photobiomodulation (PBM) and Anti-Microbial Photo Disinfection Therapy (aPDT) of Morula Stem Cells (MoSC) In-Vivo and In-Vitro

(71) Applicants: Maite Maria Cecilia Moreno Delgado, Tijuana (MX); Mary-Ellen Loretta Quintana, Downey, CA (US)

(72) Inventors: Maite Maria Cecilia Moreno Delgado, Tijuana (MX); Mary-Ellen Loretta Quintana, Downey, CA (US)

(21) Appl. No.: 18/173,026

(22) Filed: Feb. 22, 2023

Related U.S. Application Data
(60) Provisional application No. 63/312,834, filed on Feb. 22, 2022.

(51) Int. Cl. A61L 27/38

(52) U.S. Cl. CPC A61L 27/3895; A61L 27/3834; A61L 2430/12; A61L 2400/06

(57) ABSTRACT

The systems and methods herein are directed to photo-biomodulation (PBM) and anti-microbial photo disinfection therapy, aPDT, a low laser light therapy (LLLT) of morula stem cells in gingivae (GMoSC) in-vivo and in-vitro for autologous and/or allogenic applications in tissue and organ engineering with or without exosomes. The morula stem cells are initially found in gingivae in a clustered state where their proliferation capabilities are preserved through life, suitable for autologous and/or allogenic applications. The morula stem cells in gingivae tissues in the imperceptible suture of maxillae/pre-maxillae in a clustered state as a coating of gingivae tissues have been treated with PBM, aPDT, or LLLT, thereby clustered state is laser excited, which allows the morula stem cells to be imbued with a first proliferation effect and a second proliferation effect.

### CROSS-REFERENCE INFORMATION

This application claims priority from U.S. Provisional Application Ser. No. 63/312,834 filed on Feb. 22, 2022, which is hereby incorporated herein by reference in its entirety.

### FIELD OF INVENTION

The invention pertains to the field of laser assisted primitive, morula stem cells (laMoSC) existing in an imperceptible juncture of the gingivae that corresponds to the invisible sutures of the pre-maxillae and maxillary processes of a human skull. The invention teaches photobiomodulation (PBM) and Low Laser Light therapy (LLLT) with anti-microbial photo disinfection therapy, aPDT, of laser assisted entrapped morula stem cells located as a cluster in gingivae tissues, in-vivo and in-vitro.

### BACKGROUND OF THE INVENTION

Regenerative health including: growing periodontium, growing dental bone, growing new teeth, growing muscle, growing tendons, growing neural tissue; regeneration of dermatological tissues, collagen; stimulating hair growth; relieving pain; preventing and reversing arthritis and other auto-immune degenerative diseases; preventing continued hearing loss, preventing orthopedic impairments such as tendonitis, bursitis; curing cancer, treating, and curing Sars-CoV-2 and other viruses.

The systems and methods herein involve low laser light therapy and photobiomodulation. (PBM) The low laser light therapy (LLLT) provides the anti-microbial photo disinfection therapy, aPDT, in-vivo and in-vitro. PBM accelerates the separation of the morula stem cells from gingivae tissues and their proliferation in-vitro. The systems and methods herein further comprise: the obtention, proliferation and application of morula stem cells from gingivae in vivo, and the obtention, separation and expansion of morula stems cells in-vitro, which can be directed to the fields of tissue engineering, organ engineering, tissue rejuvenation and regeneration.

### SUMMARY OF INVENTION

In an aspect, a method for cell separation and growth comprises: identifying and retrieving primitive benign morula autologous or allogenic and non-tumorgenic multi-potent cells as a biological growth platform, from gingiva tissues, wherein the biological growth platform creates cell lineages, tissues, organoids, transplanted organs, and organs; using photobiomodulation on the biological growth platform, thereby stimulating separation of morula stem cells (MoSC) from the gingiva tissue; responsive to stimulating the separation of the morula stem cells from the gingiva tissues, reproducing the morula stem cells in an environment in isolation from the gingiva tissues; promoting stimulation of paracrine mesenchymal stem cells (MSC) and via addition of platelets rich in growth factors (PRGF) and the photobiomodulation (PBM), thereby generating tissues; and adding biomaterials to the PRGF and MoSC to yield a biological mass at a junction of a patient, wherein the biological mass contains biological activity derived in part from the generated tissues.

In another aspect, the biological mass at the junction of the patient is selected from the group consisting of: (1) bone tissue around teeth or dental implants; (2) rejuvenated vaginal wall; (3) urinary tract to retard incontinence; and (4) cell specific cell lineages for deriving organs.

In another aspect, generating the tissues comprises using an allogenic ascendant to descendant transplantation in a direct line, thereby providing a structural platform within the patient to: repair arthritic/degenerative diseases, treat hearing loss, regrow lost hair, and treat mental diseases via exosomes.

In another aspect, the biomaterials is osseo-conductive material selected from silicate phosphate cement, silicate lithium porcelain, and/or calcium phosphate (CaP) bioceramic material, dicalcium sulfate, titanium, zirconium, and/or other new materials for bone grafting in the human body.

In another aspect, the morula stem cells are seeded or luted around margins within a matrix that preserves a microenvironment of molecules and proteins located at the imperceptible suture of the pre-maxillae/maxillae.

In another aspect, the teeth is in a state associated with periodontal disease or a state associated with trauma from occlusion or other bone loss in the maxillae and/or mandible.

In another aspect, the paracrine mesenchymal stem cells (MSC) maturing to the mature state construct, in part, the biological mass for treating ailments or illnesses in the patient.

In another aspect, treating the ailments or illnesses in the patient comprises: inhibiting autoimmune diseases due to the immunomodulatory capacity to enhance cell function, quantity, and quality of any cell type in identified autoimmune compromised patients; mitigating cancer progression in identified cancer patients; reversing symptoms and regenerate damaged tissues of SARS-Cov-2 and variants in identified Covid-19 patients; attenuating and/or correcting mental diseases; attenuating and/or correcting brain aneurysms; attenuating hearing loss; improving effects of aging in dermatological tissue and muscle tone; improving erectile dysfunction; inhibiting aging diseases of the eye composed of tear ducts, retina, iris, sclera and crystaline; improving recovery from orthopedic injuries and orthopedic diseases; inhibiting and curing diabetes; inhibiting and/or repairing cardio-vascular conditions; inhibiting and/or repairing diseases in hepatic systems; inhibiting and/or repairing diseases in pulmonary systems; inhibiting and/or repairing diseases in urinary systems; inhibiting and/or repairing diseases respiratory systems; inhibiting and/or repairing diseases in gastrointestinal systems; inhibiting and/or repairing diseases in endocrine systems; inhibiting and/or repairing diseases in lymphatic systems; inhibiting and/or repairing diseases in reproductive systems; inhibiting obesity; transporting medical substances to targeted systems and organs; and improving success rates of transplanted organs.

In another aspect, wherein the cell specific cell lineages for deriving the organs comprise the biological mass configured as a biologically active material for: creating new transplantable human organs; regrowing lost fingers; inhibiting of dental pulp tooth degeneration in the patient; growing new teeth in the patient; and inhibiting hair loss and supporting new cranial hair growth in the patient; growing periodontium in the patient; growing dental bone in the patient; growing muscle in the patient; growing tendons in the patient; growing neural tissue in the patient; regenerating dermatological tissues in the patient; and regenerating collagen in the patient.

In another aspect, inhibiting of the dental pulp tooth degeneration in the patient comprises: promoting the elimination of root canal treatment in the patient having necrotic pulp and apical fenestration on the cortical of the maxillae/mandible; promoting elimination of inflammation in the patient with inflammatory process of the dental pulp due to decay wherein the dental pulp is vital; and promoting sealed dentinal tubules wherein the pulp is vital via photobiomodulation.

In another aspect, growing the new teeth in the patient comprises: promoting the new tooth growth in the patient who has lost a tooth in the maxillae or mandible having a dormant dental follicle generating a supernumerary tooth in-vivo; creating a mix of bone bond and particles of bone with the morula stem cells in the proportion of 0.2-0.8 cubic centimeter (cc) of bone bond and 0.2-0.8 cc particles of bone at 50/50 proportion with 10-15 drops of the morula stem cells to create a thick paste molded with a hand dental instrument during surgery into the existing defect within the junction of the patient and sutured shut with nylon 3-0 caliber stitches, thereby stitches remain in place for 12-20 days and are configured to be removed by a dentist; and injecting the morula stem cells into a maxillae or mandible area associated with tooth loss, wherein exosomes of adipose tissue extraction are administered to the treatment area via inhalation method for approximately 15-50 minutes and photobiomodulation at a 905 nm or 976 nm wavelength at 0.6-1.0 Joules/square centimeter administered to the treatment area after inhalation within 1-2 hours, wherein the exosomes are allogenic or autologous.

In another aspect, inhibiting the hair loss and supporting the new cranial hair growth comprises: injecting 2 drops of saline solution containing approximately 4,000-5,000 morula stem cells into the cranial dermis where hair loss is observed; and using a syringe type device to perforate derma at 0.25-0.50 mm depth, wherein the syringe type device contains a saline solution with morula stem cells in an allogenic and/or autogenic state, thereby administering: (1) multiple injections of the saline solution and (2) exosomes of adipose tissue extraction to a treatment area via an inhalation method for approximately 15-50 minutes and photobiomodulation at 905 nm or 976 nm wavelength at 0.6-1.0 Joules/square centimeter administered to the treatment area after the inhalation within 1-2 hours, wherein the exosomes are allogenic or autologous.

In an aspect, a biological mass at a junction of a patient comprising: paracrine mesenchymal stem cells (MSC) stimulated to a mature state from morula stem cells, via addition of platelets rich in growth factors (PRGF) and photobiomodulation; and biomaterials added to the PRGF such that the MSC form a biological mass at a junction of a patient, wherein the biomaterials is osseo-conductive material selected from silicate phosphate cement, silicate lithium porcelain, and/or calcium phosphate (CaP) bioceramic material, dicalcium sulfate, titanium, zirconium, and/or other new materials for bone grafting in the human body.

In another aspect, the biological mass further comprises exosomes of MoSC or adipose tissue extraction, wherein the exosomes are allogenic or autologous.

In an aspect, a consumer or medical product comprises: isolated morula stem cells (MoSC), and/or exosomes, and excipient ingredients, wherein the consumer or medical products are: pharmaceutical products, medical and veterinary preparations, hygiene and sanitary products for medical use, foods and dietary substances of medical and veterinary use, baby food, food supplements for humans and mammals, dressing materials and poultice, dental impression materials and dental liners, or dental cements, disinfectants, products to eliminate harming animals, and fungicides.

### BRIEF DESCRIPTION OF DRAWINGS

The present invention, in accordance with one or more various embodiments, is described in detail with reference to the following figures. The drawings are provided for purposes of illustration only and merely depict typical uses or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

Some of the figures included herein illustrate various embodiments of the invention from different viewing angles. Although the accompanying descriptive text may refer to such views as “top,” “bottom” or “side” views, such references are merely descriptive and do not imply or require that the invention be implemented or used in a particular spatial orientation unless explicitly stated otherwise.

The figures which accompany the written portion of this specification illustrate embodiments and method(s) of use for the present invention.

FIG. 1 is an image which corresponds to the morula cells, wherein the morula cells are marked by tinting SOX2 (green), E-CADHERIN (magenta), β-CATENIN (red), GATA3 (magenta), PARD6 (green), F-ACTIN (red), and DAPI (blue) in human embryos.

FIGS. 2A, 2B, 2C, 2D, 2E, 2F, 2G, 2H, and 2I are depictions of the morula stem cells applicated for dental patients.

FIG. 3 is an image of mesenchymal stem cell application.

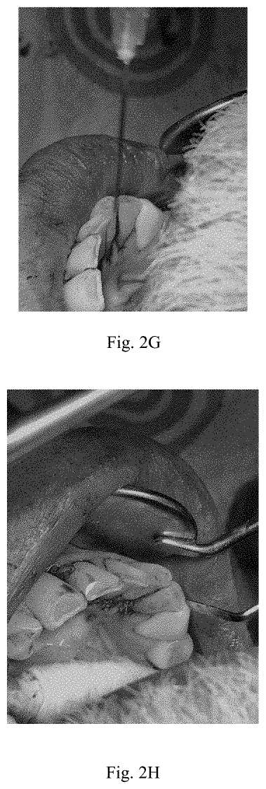

FIG. 4 is a heart patch seeded with gingivae mesenchymal stem cells produced in The Netherlands 2017. The second image shows the differentiation to myocardial cells in the heart as it beats.

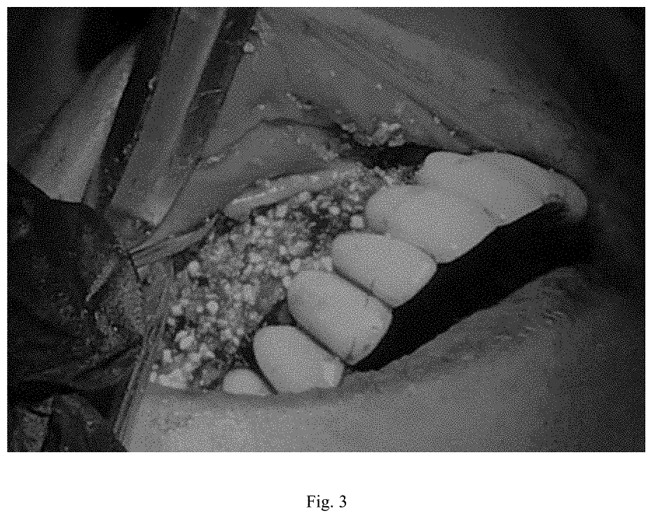

FIG. 5A is a depiction of the primitive stem cells being expanded in vitro.

FIG. 5B is a depiction of dental pulp mesenchymal stem cells in isolation in vitro.

FIG. 6A is a depiction of the application of stem cells in a dental practice.

FIG. 6B is CT scan corresponding to FIG. 6a.

FIG. 7 is a depiction of isolation of stem cells from gingiva.

FIGS. 8A, 8B, 8C, 8D, 8E, 8F, 8G, 8H, and 8I are depictions of the application of stem cells in a dental practice and the corresponding CT scan, respectively.

FIG. 9 is a depiction of isolated morula stem cells from gingiva under a microscope, wherein the isolated morula stem cells were originally identified as primitive benign stem cells in 2018.

### DETAILED DESCRIPTION OF THE EMBODIMENTS OF THE INVENTION

From time-to-time, the present invention is described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative medical and dental environments.

Reference is also made to the figures, as presented herein. The figures are not intended to be exhaustive or to limit the invention to the precise form disclosed. It should be understood that the invention can be practiced with modification and alteration, and that the invention be limited only by the claims and the equivalents thereof.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

Gingivae, as used herein, is defined as the pink tissue that surrounds and protects the teeth. It seals each tooth into its alveolus. Macroscopically, it consists of a keratinized area (soft pink) called attached gingivae and a non-keratinized (red and vascularized) area called free gingiva. Gingivae is part of the support of periodontal tissues in the mouth which include periodontal ligaments, cementum and the socket alveoli bone. Gingivae tissue is a layer that coats the periosteum and the maxillary and mandible bones. Between two teeth is the papillae part of the gingivae and its size can vary according to the size of the teeth it adheres to. Each tooth has what is called the free gingivae margin or junction around it and it ends at the sulcular crevice part of the gingivae that seals the tooth to its alveolus, such as an imperceptible suture. Normally, the size of the free gingivae margin measures up to 3 mm. Microscopically, gingivae consist of cells (5%), collagen (65%), and fibrous tissues (30%). The junctional epithelium of the gingivae is strategically located at the sulcular crevice and helps to prevent microbial diseases. Lastly, the gingivae mediate the innate immune response to infectious microbial biofilm inflammation.

Mesenchymal stem cells, as used herein, as opposed to primitive, benign morula stem cells, are defined as mature stem cells. The mesenchymal stem cells or mature stem cells are undifferentiated cells that can differentiate into several different kinds of cells in the human body. Mesenchymal stem cells are specialized cells, which: replicate and give rise to daughter cells and other cells that further differentiate into a variety of tissues. Mesenchymal stem cells can divide through mitosis and produce a continuous supply of stem cells which diminishes with aging.

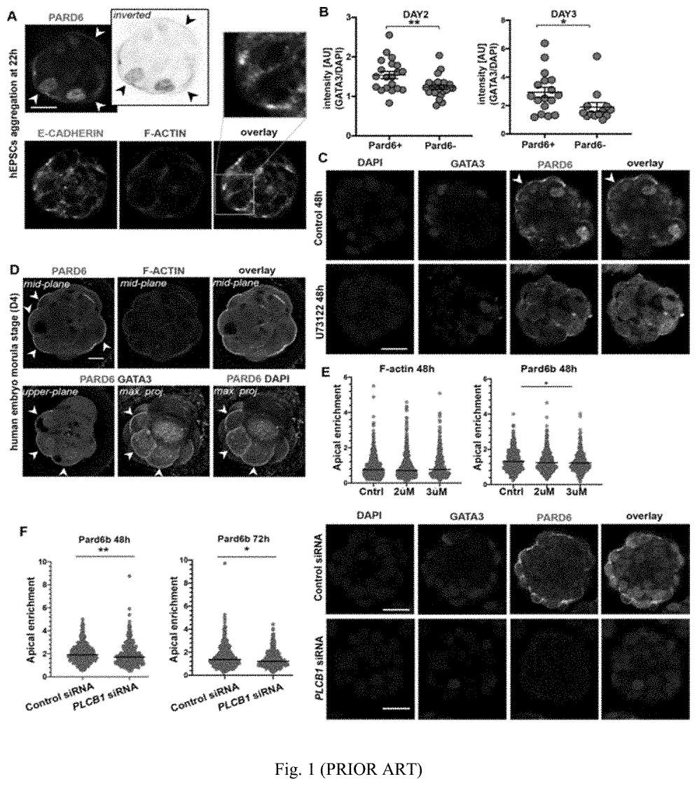

Morula stem cells or primitive stem cells are pluripotent stem cells found in embryonic tissue and are the main area of interest in this patent as they are also found in the gingivae and seen in the systems and methods herein as undifferentiated primitive benign stem cells. (See FIG. 1, 4, or 9.) Being primitive stem cells, morula cells do not include telomeres in their replications throughout life. All other stem cells, such as mesenchymal stem cells, are mature stem cells which while undifferentiated, have senescence and can become tumorigenic. Morula stem cells can be expanded and applied in regenerative medical and stemmatological procedures and purposes, as disclosed below in the systems and methods herein.

Morula stem cells form only on days 3 to 4.5 of conception and live only in the gingivae, which is the area of interest in the systems and methods herein. Morula stem cells do not experience senescence and do not change throughout life in the gingivae. Mesenchymal stem cells evolved after the mesoderm formed during embryonic growth occurring the sixth week after conception and at different times during maturation to adulthood. Every 6 to 10 years, the cells in the human body renew themselves with differentiated mesenchymal stem cells. For example, the bones of the skeleton are replaced every 6 years.

Note, the stimulated morula cells applied in patients also led to other clinical outcomes, such as reduced arthritis in human hands. After two hours, the thumbs in particular of both human hands had reduced pain and improved movement.

Exosomes, as used herein, are extracellular vesicles (EV) secreted by all cell types and contain micro-RNA and other biomarkers from their originating cell. These EV can transfer molecules from one morula stem cell to another cell via membrane vesicle trafficking, thereby influencing new chemical reactions and behaviors in cells in a process known as quantum synergy. Morula exosomes present in gingivae provide pluripotent, non-tumorigenic, anti-inflammatory, immunomodulatory capacities, and have the potential to restore human hearts, livers, brains, eyes, bones, muscles, teeth, periodontium, joints, vessels, hair, hearing, and help healing or improve chronic and acute diseases, suppress cancer, and promote tissue and/or organ engineering.

The transcriptome, as used herein, is an array of RNA transcripts (messenger RNA and noncoding RNA) produced in an originating morula stem cell. The array of RNA transcripts undergo transcription at specific development stages and/or present at physiological conditions within a cell type or tissue, i.e., a process of copying a segment of DNA into RNA wherein the RNA is encoded into proteins.

The systems and methods herein are directed to laser assisted morula stem cells (laMoSC) (as a sub-niche or entrapped cluster of cells) in an imperceptive junction in the area of interest of gingivae, that corresponds to the suture of the premaxillae and maxillary processes of a human skull, which are treated with photobiomodulation and aPDT of the gingivae tissues as methods of obtention in-vitro and in-vivo of MoSC and mesenchymal stem cells (MSC), utilized for, but not limited to the following: tissue engineering, organ engineering, organoids engineering, transplants, and regeneration of tissues.

In contrast to techniques and teachings in the field of art, the systems and methods herein are directed to a newly discovered stem cell (i.e., stem cell of use) in the gingivae that is unlike all other stem cells in the human body or any other mammal. The stem cell of use is a precursor cell existing in embryological development, between the zygote and the blastocyst. This existential placement makes the stem cell of use, which is referred to as the morula, i.e., a primitive, benign, pluripotent stem cell. The morula stem cell(s) are formed on day 3-4.5 in the life cycle of an embryo. These morula stem cells continue to exist as a sub-cluster within gingivae in a particular location of the pre-maxillae\maxillae and these morula stem cells continue to form throughout human life without senescence. These cells, unlike all other pluripotent stem cells (embryonic chord) are easy to extract from a patient, in a pain free manner in the outpatient setting of a dentist's chair. Applying laser irradiation promotes photobiomodulation (PBM) and anti-microbial photo disinfection therapy (aPDT) as an integral part of the process to separate the morula stem cells from the gingivae and provide disinfection, promote proliferation and rapid expansion of the morula stem cells of the systems and methods herein. However, the discovery of the existence of gingivae morula stem cells is primary to the systems and methods herein. In the systems and methods herein, morula gingivae stem cells irradiated twice in-vitro and twice in-vivo in clinical patients with 905 nm wavelength or 976 nm wavelength have grown at a faster rate and in greater numbers than gingivae or mesenchymal stem cells without laser irradiation.

In further contrast to techniques and teachings in the field of art, the systems and methods herein are directed to: (i) the extraction of morula stem cells and (ii) in-vitro and in-vivo patient treatments. The morula stem cells are disinfected before and after extraction from patients to avoid microbial contamination and ensure purity. Therefore, the dentist or physician assistant conducting the biopsy of the gingivae in the area of interest uses a very specific PBM parameter while extracting the tissues and a specified PBM parameter before the biopsy of the tissues and the start of cell processing. The systems and methods herein involve: (i) disinfection when a specific laser wavelength is applied before the biopsy is performed and a specified laser disinfection in the laboratory in cultures with the extracted or biopsied tissues, and (ii) photo biomodulation that allows the morula stem cells to reproduce and separate faster, with greater adenosine triphosphate (ATP) quantities than that of non-laser treated stem cells.

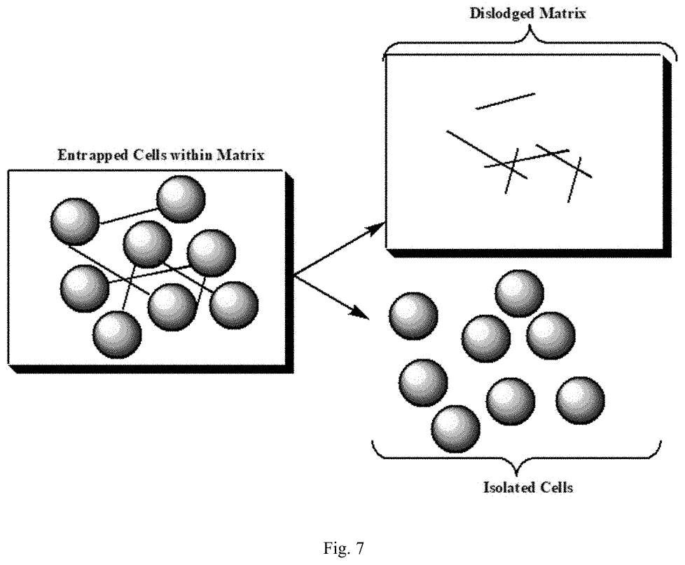

As depicted in FIG. 7, morula stem cells within the gingivae are entrapped cells within a biological matrix, such as tissue matter, wherein the morula stem cells adhere to gingiva and potentially to each other via connective pathways (e.g., biological matter) to construct the biological matrix that coincides with the pre-maxillae/maxillae junction. The applied laser wavelength of the systems and methods herein can have sufficiently enough energy to sever the connective pathways within the biological matrix, thereby leading to: (1) a dislodged matrix composed of its communicative, signaling pathways (i.e., gingiva absent of morula stem cells); and (2) isolated morula stem cells, wherein the isolated morula stem cells (i.e., morula stem cells in an isolated state) are of a higher purity or of a state configured for easier purification. Stated another way, the entrapped morula stem cells, which were newly identified by Maite Moreno, DDS, in gingivae can be laser assisted and treated with biological solution(s), thereby leading to morula stem cells in an isolated state which undergoes an initial first proliferation effect and a subsequent second proliferation effect. More particularly, the biological solutions (e.g., PBS, saline solution, basal media, growth media, culture media, and differentiation media) can contact gingivae tissues, thereby separating morula stem cells from gingivae tissues. Upon separation of the morula stem cell from the gingivae tissue, the gingivae is discarded. The morula stem cells of the systems and methods herein can be delivered on the scale of million cells with 98% or viability in saline solution in a sterile syringe of 10 ml.

The isolated morula stem cells are able to achieve a second proliferation effect, which can be a more rapid and more efficient proliferation than the first proliferation effect in, for example, a petri dish or flask, wherein the proliferation effect is the capability of the morula stem cells to: (1) grow rapidly in number; and (2) evolve into more complex biological cell structures, such as mesenchymal stem cells (MSC) or any other cell. More particularly, the laser can provide photo-excitation frequencies which transfer energy to organelles within the morula stem cells in the isolated state. Stated another way, the organelles of the isolated morula stem cells are in an exposed state that can receive the energy transfer which lead to organelles in an excited state being able to achieve more rapid and efficient proliferation. Organelles, which contain proteins, DNA, and other biological entities, can be functionally altered when in the excited state due to photo-excitation from PBM or aPDT, i.e., photo-excitation frequencies contact the organelles, which contain proteins, DNA, and other biological entities for energy transfer. The morula stem cells (MoSc) and/or mesenchymal stem cells (MSC) can be combined with biomaterials capable of forming new tissue or replenishing lost tissue in a patient. More particularly, the entrapped morula stem cells within the matrix correspond to a cluster that is localized after different divisions of the embryo in the area of interest, such as the suture (i.e., coating for new maxillae formation) within the gingivae tissue and encapsulated by the 8th week of conception when the maxillae forms, wherein the morula stem cells remain throughout the life of the human or mammal. Additionally, the morula stem cells are seeded or luted within the matrix that preserves the microenvironment of the molecules and proteins which is located at the imperceptible suture of the pre-maxillae/maxillae.

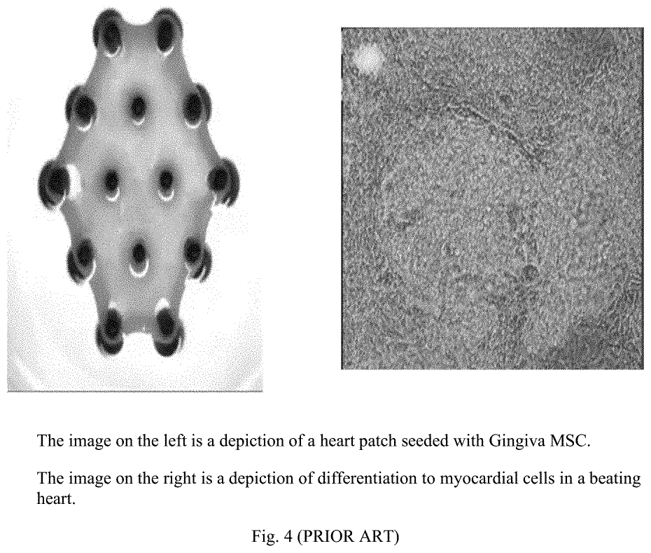

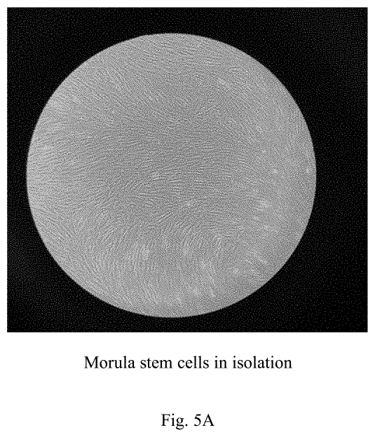

In the systems and methods herein, the biological mass formed by morula stem cells, isolated from the entrapped matrix as seen in FIG. 5A and diagrammed in FIG. 7, can be applied in a variety of tissues using autologous graphs in humans and mammals, from which regenerative tissue will start to form along with paracrine mesenchymal stem cells in vivo. For example, in the treatment of a patient(s) at a junction in the patient, the biological mass is derived from said morula stem cells of the systems and methods herein, wherein said morula stem cells are used as a structural platform in the patient for constructing: (1) functional bone material configured for dental applications, such as bone tissue around teeth or dental implants; (2) reinvented vaginal wall in postmenopausal women; (3) urinary tract mesh repair to inhibit incontinence; and (4) cell specific cell lineages for deriving organs. Additional examples, in the treatment of a patient(s) at a junction in the patient, the regenerative tissue, as generated using an allogenic ascendant to descendant transplantation in a direct line, can be used as a structural platform in the patient for (1) repairing arthritic/degenerative diseases, such as repairing lower discs in the spine, prior or post-operative, through application(s) of expanded morula stem cells, (2) treating PTSD and other mental diseases through morula exosomes, (3) improving any disease, such as lost hearing, through the application of a deceased parent's thawed morula stem cells, and (4) improving and regrowing lost hair through application of morula stem cells and exosomes, in addition to several other regenerative health outcomes.

In the systems and methods herein, regenerative health outcomes include: growing periodontium, growing dental bone, growing new teeth, growing muscle, growing tendons, growing neural tissue; regeneration of dermatological tissues, collagen; stimulating hair growth; relieving pain; preventing and reversing arthritis and other auto-immune degenerative diseases; preventing continued hearing loss, preventing orthopedic impairments such as tendonitis, bursitis; curing cancer, treating, and curing Sars-Cov-2 and other viruses.

In the systems and methods herein, the structural platform in the patient can be derived from: (1) morula stem cells, as isolated from the gingivae tissues; (2) mesenchymal stem cells; (3) exosomes in combination with morula stem cells, as isolated from the gingivae tissue; (4) exosomes in combination with mesenchymal stem cells; (5) morula stem cells, as isolated from the gingivae tissues, in combination with mesenchymal stem cells; and/or (6) morula stem cells, as isolated from the gingivae tissues, in combination with mesenchymal stem cells and exosomes. The structural platform can be combined with biomaterials and/or platelets rich in growth factors (PRGF) to derive regenerative tissue from mesenchymal stem cell tissues (MSC) and biological masses at the junction of a patient from morula stem cells (MoSC), as isolated from the gingivae tissue.

Stated another way, the systems and methods herein are directed to a “cellument”, i.e., a combination of dental and medical knowledge with specific laser frequencies used to produce highly potent, cloned autologous and allogenic morula stem cells isolated from gingivae tissue, wherein the morula stem cells isolated gingivae tissue can be applicable in regenerative medicine, general medicine (pain reduction, anti-inflammatory treatments, etc.) including cures for a multitude of diseases such as Sars-CoV-2, diabetes, all auto-immune diseases, and gastro-intestinal diseases, such as Crohn's and IBS, and cancer. More particularly, laser-assisted techniques (PBM and aPDT) for obtention of morula stem cells found in the gingivae, result in morula stem cells in isolated state, wherein the morula stem cells in the isolated state are pluripotent primitive benign stem cells that have the potentiality to rebirth body parts because they are the cells of a recently evolved zygote. Laser assisted morula stem cells in the isolated state can replicate and perform the functions of a forming embryo. More particularly, the morula stem cells (MoSC) in the isolated state can restore function to cells by repairing signaling and damaged pathways to neurons, tendons, muscles, etc. Exosomes within MoSC can possess specific membrane-associated proteins, such as CD9, CD63, CD81, and tumor suppressor genes, which can regulate: (1) levels of gene expression in physiological environments, (2) chromatin architecture/epigenetic memory, (3) transcription, (4) RNA splicing, (5) editing, (6) translation, and (7) turnover.

In the systems and methods herein, MoSC in the isolated state can diminish pain and regulate cellular metabolic activity including mitigating the natural aging processes and promoting balance between reactive oxygen species (ROS), the byproducts of cellular oxidative metabolism and other senescent factors that cause bodies to break down especially over time. More particularly, the absorption of photons, i.e., photoexcitation, by MoSC in the isolated state, MSC, and exosomes cause organelles therein to have electrons pumped into an excited state from a ground state. In the excited state, redox and peroxidation reaction pathways can be activated within the MoSC in the isolated state, MSC, and exosomes. The ROS is derived from oxygen to make an active oxidant, i.e., an activated molecule, which reacts with the lipids, proteins, organelles, and other systems. Water in the MoSC in the isolated state, MSC, and exosomes can quench the ROS, which brings the organelles back to a ground state. The ROS within the MoSC in the isolated state, MSC, and exosomes of the systems and methods herein, can be modulated by anti-microbial byproducts or lysosomal enzymes. Despite the MoSC in the isolated state, MSC, and exosomes of the systems and methods herein having ROS of high oxidative capability, the ROS have short radical lifetimes, small active distance scales, selectivity for anionic microbes, and resistance to oxidative stress of mammalian cells to minimize damage to neighboring host tissues.

In the systems and methods herein, the MoSC in the isolated state are delivered to a site in the body such that the paracrine system of each targeted tissue is activated with other dormant stem cells in the area of interest. Thereafter, the MoSC in the isolated state work to produce beneficial repair, disinflammation, reduce pain, numbness and facilitate the recovery of the normal function. Being pluripotent, the laser assisted MoSC in the isolated state have biomodulator capacities that are demonstrated in a period of two hours to approximately 5 years, which have uses that can include, but are not limited, to treating the following ailments and illnesses in patients: inhibiting autoimmune diseases due to the immunomodulatory capacity to enhance cell function, quantity and quality of any cell type in identified autoimmune compromised patients; mitigating cancer progression in identified cancer patients; reversing symptoms and regenerating damaged tissues of SARS-CoV-2 and variants in identified Covid-19 patients; attenuating and/or correcting mental diseases; attenuating and/or correcting brain aneurysms; attenuating hearing loss; improving effects of aging in dermatological tissue and muscle tone; improving erectile dysfunction; inhibiting aging diseases of the eye which is composed of sclera, cornea, iris, pupil, lens, retina, optic nerve, tear ducts, retina, iris, sclera, and crystalline lens; improving recovery from orthopedic injuries and orthopedic diseases; inhibiting and curing diabetes; inhibiting and/or repairing cardio-vascular conditions; inhibiting and/or repairing diseases in hepatic systems; inhibiting and/or repairing diseases in pulmonary systems; inhibiting and/or repairing diseases in urinary systems; inhibiting and/or repairing diseases respiratory systems; inhibiting and/or repairing diseases in gastrointestinal systems; inhibiting and/or repairing diseases in endocrine systems; inhibiting and/or repairing diseases in lymphatic systems; inhibiting and/or repairing diseases in reproductive systems; inhibiting obesity; transporting medical substances to targeted systems and organs; and improving success rates of transplanted organs using an ascendent or descendent line.

The systems and methods herein, further comprise exosomes. Particular PBM and aPDT can be used to obtain the fluence in morula stem cells, such as the morula stem cells in the isolated state, which can implement a successful conversion to any other wavelengths used with other laser equipment. Exosomes irradiated using the PBM and aPDT parameters described, benefit in the same manner as these benign primitive stem cells. Exosomes can be instrumental in the growth of tissue and bone in a real dental practice; hair growth; dermal regeneration; and pain reduction for arthritic joints. Exosomes from gingiva comprehend families of EVs that impact multiple systems. In dentistry, the exosomes are noted to enhance bone mineralization and disinflammation of periodontal ligaments. It has been noted that gingiva derived exosomes provide anti-inflammatory and immune response and affect neuron development and differentiation; negative regulation of neuron apoptotic process and in vasculogenesis; and regulation of angiogenesis (TGF-β, BMPs, and GDFs). The transcripts in the exosomes of the systems and methods herein can encode for Interleukin, Transforming TBF-β, WNT, and growth factor protein families. The proteins as listed below are implicated in biological processes associated with biological activity of cells, organs, and other living entities.

More particularly, the transcripts in the exosomes of the systems and methods herein can include IL19, IL37, IL21, IL17A, IL15, IL12A, IL12B, IL6, IL7, IL5, IL25, IL27, IL32, IL1B, IL36B, IL16, IL36G, and IL33 for encoding the respective proteins of INTERLEUKIN 19, INTERLEUKIN 37, INTERLEUKIN 21, INTERLEUKIN 17A, INTERLEUKIN 15, INTERLEUKIN 12A, INTERLEUKIN 12B, INTERLEUKIN 6, INTERLEUKIN 7, INTERLEUKIN 5, INTERLEUKIN 25, INTERLEUKIN 27, INTERLEUKIN 32, INTERLEUKIN 1B, INTERLEUKIN 36B, INTERLEUKIN 16, INTERLEUKIN 36G, and INTERLEUKIN 33.

More particularly, the transcripts in the exosomes of the systems and methods herein can include TGFB1, TGFB2, TGFB3, BMP7, AMH, GDF9, BMP4, BMP3, BMP8A, BMP8B, BMP6, GDF3, GDF2, GDF1, GDF6, GDFS, BMP2, BMP1, BMP10, BMP5, BMP15, GDF15, GDF11, GDF10, INHA, INHBA, and INHBC for encoding the respective proteins of Transforming Growth Factor Beta 1, Transforming Growth Factor Beta 2, Transforming Growth Factor Beta 3, Bone Morphogenetic Protein 7, Anti-Mullerian Hormone, Growth Differentiation Factor 9, Bone Morphogenetic Protein 4, Bone Morphogenetic Protein 3, Bone Morphogenetic Protein 8a, Bone Morphogenetic Protein 8b, Bone Morphogenetic Protein 6, Growth Differentiation Factor 3, Growth Differentiation Factor 2, Growth Differentiation Factor 1, Growth Differentiation Factor 6, Growth Differentiation Factor 5, Bone Morphogenetic Protein 2, Bone Morphogenetic Protein 1, Bone Morphogenetic Protein 10, Bone Morphogenetic Protein 5, Bone Morphogenetic Protein 15, Growth Differentiation Factor 15, Growth Differentiation Factor 11, Growth Differentiation Factor 10, Inhibin Subunit alpha, Inhibin Subunit Beta A, and Inhibin Subunit Beta C.

More particularly, the transcripts in the exosomes of the systems and methods herein can include WNT2B, WNT3, WNT10A, WNT10B, WNT4, WNT8A, WNT9A, WNT9B, WNT16, WNT7A, WNT11, WNT5B, and WNT5A for encoding the respective proteins of WNT FAMILY MEMBER 2B, WNT FAMILY MEMBER 3, WNT FAMILY MEMBER 10A, WNT FAMILY MEMBER 10B, WNT FAMILY MEMBER 4, WNT FAMILY MEMBER 8A, WNT FAMILY MEMBER 9A, WNT FAMILY MEMBER 9B, WNT FAMILY MEMBER 16, WNT FAMILY MEMBER 7A, WNT FAMILY MEMBER 11, WNT FAMILY MEMBER 5B, and WNT FAMILY MEMBER 5A.

More particularly, the transcripts in the exosomes of the systems and methods herein can include FGF10, FGF5, FGF2, FGF13, FGF19, FGF18, FGF1, FGF9, FGF12, FGF7, FGF6, FGF11, FGF4, FGF23, FGF20, FGF14, PSPN, GDNF, PGF, VEGFA, VEGFC, VEGFB, NGF, NTF3, NTF4, and BDNF for encoding the respective proteins of FIBROBLAST GROWTH FACTOR 10, FIBROBLAST GROWTH FACTOR 5, FIBROBLAST GROWTH FACTOR 2, FIBROBLAST GROWTH FACTOR 13, FIBROBLAST GROWTH FACTOR 19, FIBROBLAST GROWTH FACTOR 18, FIBROBLAST GROWTH FACTOR 1, FIBROBLAST GROWTH FACTOR 9, FIBROBLAST GROWTH FACTOR 12, FIBROBLAST GROWTH FACTOR 7, FIBROBLAST GROWTH FACTOR 6, FIBROBLAST GROWTH FACTOR 11, FIBROBLAST GROWTH FACTOR 4, FIBROBLAST GROWTH FACTOR 23, FIBROBLAST GROWTH FACTOR 20, FIBROBLAST GROWTH FACTOR 14, PERSEPHIN, GLIAL CELL DERIVED NEUROTROPHIC FACTOR, PLACENTAL GROWTH FACTOR, VASCULAR ENDOTHELIAL GROWTH FACTOR A, VASCULAR ENDOTHELIAL GROWTH FACTOR B, NERVE GROWTH FACTOR, NEUROTROPHIN 3, NEUROTROPHIN 4, and BRAIN DERIVED NEUROTROPHIC FACTOR.

In the systems and methods herein, exosomes from gingivae MSC (i.e., MSC derived directly or indirectly from human gingivae) can suppress the inflammatory response of periodontal ligament stem cells (PDLSCs) by regulating the expression of NF-κB signaling and Wnt5a, thereby the exosomes can deliver RNA for translating NF-κB as a therapeutic approach for periodontitis. More particularly, human gingiva-derived mesenchymal stem cells (hGMSCs) can be isolated from the gingival propria with regenerative, immunomodulatory, and anti-inflammatory properties. In the systems and methods herein, MSC can have therapeutic potentials of nerve regeneration and skin disorders in various types such as the cell itself, cell-free conditioned medium, or extracellular vesicles (EVs). However, the mechanobiological behavior of MSC in the system and methods herein can be closely related to the culture conditions and laser treatment conditions during the obtention of morula stem cells from gingivae, wherein the morula stem cells are in an isolated state. In the systems and methods herein, MSC in 2D and 3D infusion may possess therapeutic effects in the treatment of psoriasis, reduce the levels of Th1- and Th17-related cytokines IFN-γ, TNF-α, IL-6, IL-17A, IL-17F, IL-21, and IL-22; and upregulate the percentage of spleen CD25+CD3+ T cells while downregulating the percentage of spleen IL-17+CD3+ T cells.

In the systems and methods herein, the isolated MoSC, MSC, and/or exosomes can be contained, for example, by encapsulation or formulation with excipient ingredient(s) in consumer or medical products, wherein the consumer products or medical products comprise: pharmaceutical products, medical and veterinary preparations, hygiene and sanitary products for medical use, foods and dietary substances of medical and veterinary use, baby food, food supplements for humans and mammals, dressing materials and poultice, dental impression materials and dental liners, or dental cements, disinfectants, products to eliminate harming animals, and fungicides.

Below are exemplary examples of the systems and methods herein that have been reduced to practice. These are not an exhaustive list of examples, which are not to be construed as being confined to only these examples.

### Example 1—Tissue Sample Treatment

The systems and methods herein, lead to a simplification of and decrease in the time and cost of producing high quality allogenic and autologous morula stem cells. Prior laser methods in general dentistry have been applied for disinfection and mitochondrial stimulation. There was a natural progression to use low laser light therapy (LLLT) in implant dental surgery, teeth extracts, bone grafting, periodontal treatments, and other improvements of stomatological functions. As proficiency in dental prolotherapy techniques advanced, LLLT was applied to blood and tissues, in combination with the protocol of disinfection and protocol of proliferation, wherein the protocol of proliferation led to surprising and unexpected results. More specifically, the protocol of proliferation of the systems and methods herein enhanced the immunomodulatory regenerative capacity in patients resulting in the growth and regeneration of bone, collagen, hair, dermal tissues, cartilage, nerves, pain relief, as well as the improvement of hearing loss for 90% of patients treated for one or more of these conditions. When there was no application of LLLT and lasering of gingivae, the morula stem cells did not persist. Stated another way, the morula stem cells did not remain in a state that would have been amenable for: 1) transport to the laboratory, and 2) subsequent regenerative application in patients.

The application of LLLT and lasering of gingivae specifically involved: two gum tissue samples obtained with a tissue punch of 2.5 mm after being subjected to irradiation with 904 nm wavelength for 30 seconds at 4000 Hz, 0.5 Watts (Lasertech Mod. KVT 106 UP, Mexico) to photo-biomodulate and photo-disinfect the tissues. The tip of the laser was placed at a distance of 1.5 cm. The total fluence in the tissue was 0.8 Joules/sq cm. When wavelength laser was changed to 976 nm wavelength, the setting parameters were changed to 15 seconds at 4000 Hz, at 0.5 watts [Solase, Lazon medical laser, Co. LTD], the total fluence in the tissue was 0.8 Joules/sq cm fluence.

Subsequently, the treated gingivae tissue was made amenable for transport by being placed in a 6 cm round cell well and were covered with Dubelco's Modified Eagle Medium (DMEM). At a biochemistry laboratory, the tissue samples were immediately transferred to a 6 cm round cell well and were covered with (DMEM) and supplemented with 10% fetal bovine serum (FBS) and antibiotics 50 units/ml penicillin and streptomycin. The samples were incubated at 37° C. and 5% CO2. The medium was changed every third day. On the second day and the fourth day, the samples were irradiated in-vitro with 904 nm wavelength, for 15 seconds, at 2000 Hz, 0.5 Watts (Lasertech Mod. KVT 106 UP, Mexico) with 0.4 Joules/sq cm and at a distance of one and a half inch from the cell well container. Cells started to separate on day 15. Once adherent cells were observed in the well, then the gum tissue was discarded and the medium continued to be changed every third day until they reached 4.8×10 (6) with 98% viability. The cells were harvested with triple trypsine. On the second and fourth day, when using 976 nm wavelength red laser, the parameter settings vary to 3 seconds per 2000 Hz at 0.5 watts at one half inch from the cell well container with the photobiomodulation 0.5 cm tip (Lazon Medical Laser, Co. Ltd, China) with a 0.4 J/sq cm fluence.

More specifically, two gum tissue samples were obtained with a tissue punch of 2.5 mm after 30 seconds of irradiation at 904 nm, 4000 Hz, 0.5 Watts (Lasertech Mod. KVT 106 UP, Mexico) for photobiomodulation and photodisinfection of the tissues. The PBM tip of the 0.5 cm lens was placed at a distance of 1.5 cm from the tissue. The total fluence in the tissue was 4.6 Jules/sq cm. When a different wavelength laser was used, it was set at 976 nm wavelength, with the setting parameters changed to 25 seconds at 4000 Hz, 0.5 watts [Solase Lazon medical laser, Co. LTD], and the total fluence in the tissue remained 4.6 Jules/sq cm.

### Example 2—Tooth Growth

The systems and methods herein led to the promotion of new tooth growth in an identified patient who had lost a tooth in the maxillae/mandible having a dormant dental follicle generating a supernumerary tooth in-vivo; creating a mix of bone bond and particles of bone with morula stem cells in the proportion of 0.3-0.7 cubic centimeters (cc) of bone bond (e.g., 0.3, 0.4, 0.5, 0.6, and 0.7 cc of bone bond) and 0.2-0.8 cc of particles of bone (e.g., 0.2, 0.3, 0.4, 0.5, 0.6, 0.7, and 0.8 cc of bone bond) with 10-15 drops of the morula stem cells (e.g., 10, 11, 12, 13, 14, and 15 drops of the morula stem cells) to create a thick paste that can be molded with a hand dental instrument during surgery into the existing defect and sutured shut with nylon 3-0 caliber stitches, thereby stitches remain in place for 12-20 days (e.g., 12, 13, 14, 15, 16, 17, and 18 days) and are configured to be removed by a dentist; and injecting morula stem cells into a maxillae/mandible area associated with tooth loss, wherein exosomes of adipose tissue extraction, which are either allogenic or autologous, are administered to the treatment area via inhalation method for approximately 15-50 minutes (e.g., 15, 20, 25, 30, 35, 40, 45, and 50 minutes) and photobiomodulation at 905 nm or 976 nm wavelength at 0.6-1.0 Joules/square centimeter (e.g., 0.6, 0.7, 0.8, 0.9, and 1.0 Joules/square centimeter) administered to the treatment area after inhalation within 1-2 hours (e.g., 1.0, 1.2, 1.4, 1.6, 1.8, and 2.0 hours).

### Example 3—Paracrine Local Mesenchymal Stem Cells

Laser assisted morula stem cells activated the paracrine local mesenchymal stem cells of the tissues where there was damage and treatments were applied. In a dental practice setting, isolated MoSC were applied in treatments along with bone graft materials (by calcium sulfate-Bone Bond™, particles of bone Cerabone™ mixed with platelets rich in growth factors (PRGF.) Exosomes inhabiting the isolated morula stem cells also influenced faster osteoblast differentiation and bone mineralization through their communicative signaling mechanisms. Exosome vesicles (EV) contain WNT4, WNT11, WNT10B, and WNT16 which promote osteoblast differentiation and activation, as well as the inhibition of osteoblast apoptosis.

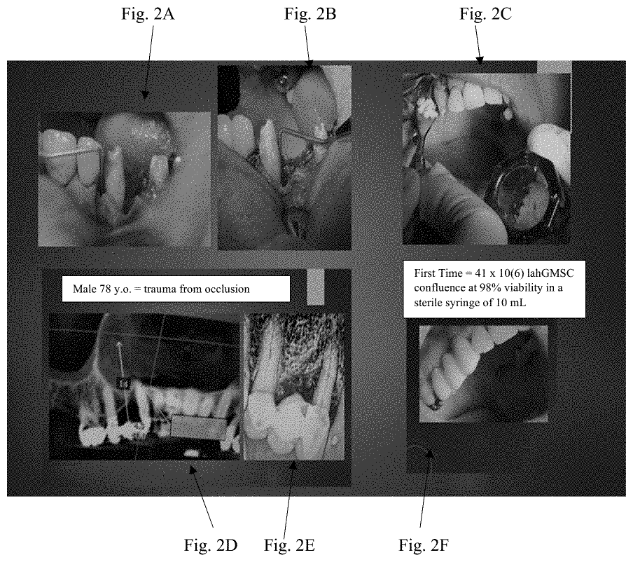

FIG. 2A: Measurement of the pocket depth in the mesial of tooth #6, 14 mm.

FIG. 2B: Measurement of the pocket depth in the distal of tooth #6, 14 mm.

FIG. 2C: Biomaterials [Bone Bond™ and bone particles] are mixed with morula stem cells and PRGF.

FIG. 2D: CT-scan showing 14 mm of pocket depth on the mesial and distal sides of the canine

FIG. 2E: 3-unit fixed bridge cemented permanently on the supporting tooth #6 and tooth #4. In this x-ray, it is possible to observe new bone formation with no infection present around tooth #6.

FIG. 2F: Clinical view of the 3-unit bridge in place.

FIG. 2G: Application of MoSC in dental patient to build bone over uncovered roots.

FIG. 2H: The biomaterials applied at the uncovered roots as a granular conglomeration will result in regenerative tissues derived from MoSC and paracrine GMSC.

### Example 3—Laser Photo Disinfection

The systems and methods herein provided a set of standard criteria that can foster a more uniform characterization of morula stem cell research and facilitate the exchange of data among investigators in multi-disciplinary interactions between the dental and medical fields. The morula tissue samples of the systems and methods herein have never been contaminated by bacterial infections in-vitro due to the methodical use of laser photo disinfection protocols.

Anti-microbial photodynamic therapy (aPDT) is a type of photo disinfection and it was used as a noninvasive modality of treatment that involves the irradiation of the tissue with a laser light and a light sensitive chromophore or photosensitizer (PS) that together pump the PS to an exited state. This action results in the elimination of any microorganisms and is an essential tool for infection control of gram-positive and gram-negative bacteria, viruses, fungi, and protozoa. APDT does not create resistance or harm tissue in spite of numerous irradiations as long as parameters are correctly followed.

The history of using photo disinfection treatments is as old as Chinese, Indian, and Egyptian cultures and may have been used longer than 1000 B.C.E. The Mayan and Aztecs (700 A.C.E.) also showed photo disinfection techniques in stelas exhibiting the healing power of the sun for some illnesses.

Moreno, M. (2008), stated that dentinal tubules were disinfected by using basic fuccine (Marking™, Mexico) as a chromophore, and infrared laser irradiation of 905 nm wavelength by tinting and eliminating bacteria in contaminated teeth. In 2013, her disinfection protocol was presented at the American College of Prosthetic Dentistry, showing decay (bacterial) disinfection and promoting sound dentine in 100% of the irradiated teeth. (Moreno M, ACP speaker forum 2013, Las Vegas Nev.).

In the last five years, there have been more than 700 research papers presented and published worldwide about the efficacy of using photo disinfection therapy in teeth and gingivae. Moreno, M. (Aachen 2018) presented how she used anti-microbial photo disinfection of dental alveoli for the extraction of teeth and the immediate placement of endosseous implants in the same appointments. (Singapore, Rome, Los Angeles 2018.)

Using low laser light therapy (LLLT), Maite Moreno used photo disinfection to decontaminate periimplantitis and necrotic bone. Moreno's method has been to apply sterilized gauze with tetracycline powder (500 mg) directly to the infected area of bone for 15 minutes and then irrigate the area with saline solution and tetracycline to remove the area of any debris in the adjacent tissues. Afterwards, disinfection was achieved through the application of low laser irradiation at 905 nm wavelength, to 4000 Hz, at 0.5 Watts power. The laser was applied at a distance of half an inch with a tip lens of 2.2 mm diameter at 30 seconds per area irradiated, covering the alveolus for a total of 2 minutes and 2 minutes in necrotic bone areas.

The instrument used was Lasertech Dental Laser LD, KVT 106 UP. Photo disinfection was also applied to the dental implant's surface area for 30 seconds on each side of the implant, and to prevent overheating of the metal, the implants were irrigated with 10 ml saline solution and 300 mg powder Tetrex to the surrounding bone. Osseo conductive materials are also used to fill the defective area, included dicalcium sulfate (Bone Bond, Mis™), particles of bovine bone (Bio-Oss™, Geitsligh™ Switzerland) with plasma rich in growth factors (PRGF), which help to make a new bone bed for new bone growth. A non-re-absorbable collagen membrane is not required. Result: in 98% of the cases there was new bone tissue grown both vertically and horizontally around the implants. It was concluded that using photo disinfection therapy (e.g., LLLT) effectively treats periimplantitis and infected socket bone and allows the immediate placement of implants.

Using these antimicrobial photodynamic therapy (aPDT) techniques, facilitated their use in-vivo and in-vitro obtention and expansion of gingivae morula stem cells. It is crucial that completely sterilized, “clean” morula stem cells be biopsied from gingivae tissue in-vivo, and delivered to the laboratory in-vitro. In the well, petri dish or flask where the tissue will be processed, there must be absolutely no contamination of any type by any microorganism.

### Example 4—Gingiva Tissue as an Entrapped Matrix Containing Stem Cells

Gingiva tissues containing morula stem cells were: obtained in Tijuana, expanded in a 5-week process, and sent to Mexico City for analysis and diagnosis of the type of cells obtained, which was deemed to be primitive benign cells. Another batch of morula stem cells were later confirmed to have precursor cells to trophoblasts. Patients of the dental practice, using the systems and methods herein, experience multi-disciplinary medical and dental benefits of having morula primitive benign autologous stem cell applications in their bodies. The clinical results were 100% positive for every patient in regeneration and improvements to their dental and general health conditions (Alpha Testing: 10 patients with 905 nm wavelength infrared laser; and Beta Testing: 2 patients with 976 mm wavelength red laser).

Two gum tissue samples were obtained with a tissue punch of 2.5 mm after subjected to infrared irradiation at 904 nm wavelength, for 30 seconds, at 4000 Hz, 0.5 Watts (Laserthec Mod. KVT 106 UP, Mexico) to biostimulate and disinfect tissues with a distance of 1.5 cm, at 0.8 Joules/sq cm, with a lens of 3 mm. Subsequently, the gingivae tissue was placed in a sterilized tube in a solution of PBS. At the biochemistry laboratory, the tissue samples were immediately placed in a 6 cm round cell well, 45 minutes previously coated with trypsin SM (at 0.025%). The gingivae tissue were covered with Dubelco's Modified Eagle Medium (DMEM) and supplemented with 10% fetal bovine serum (FBS) and antibiotics 50 units/ml penicillin. The samples were incubated at 37° C. and 5% of carbon dioxide. The medium was changed every third day. The second and fourth day the samples were irradiated once each time in vitro with infrared radiation at 904 nm wavelength, for 15 seconds, at 2000 Hz, 0.5 Watts (Laserthec Mod. KVT 106 UP, Mexico.) with 0.4 Joules/sq·cm for 15 seconds and at distance of one and half inch from the cell well container. The morula stem cells started to separate on day 14. Once the morula stem cells were observed to adhere in the well, the gum gingivae tissue was discarded. The medium was continued to be changed every third day until 3.6-4.8×106 confluence was reached in a process of five weeks of expansion. With Lazon Solase laser red irradiation at 976 nm wavelength the intraoral hand piece was used with a lens of 8 mm. The distance from the lens to the tissue was kept the same as before. The parameters were the same as before, at 0.5 Watts at 0.8 Joules/sq cm and only the time changed to 20 seconds. And the in vitro irradiation at 0.4 Joules/sq/cm penetration varied to 5 seconds application each time. In every laser in vitro application (with any of the two lasers used), the tip of the laser was placed at a distance of an inch and a half away from the tissue.

The stem cells extracted and used in the systems and methods herein, were gingivae laser assisted morula stem cells (laMoSC). The systems and methods herein are separating and expanding stem cells but not reprogramming or inducing stem cells. However, future directions may involve: (1) induction of morula stem cells in new cell line lineages; (2) investigating cells for obtention or deriving organelles or higher level organs during applications of mesenchymal stem cells; and (3) additions to hyaluronic acid treatments, collagen treatments, chitosan scaffolds, cream solutions, ointments, new dental material compounds, disinfectants, baby foods, veterinary materials, and ointments and treatments within consumer products.

### Example 5—Morula Stem Cells and Gingivae Mesenchymal Stem Cells

The systems and methods herein can use stimulated morula stem cells (MoSC) and gingivae-derived mesenchymal stem cells (MSC). The different patterns depicted in the image of FIGS. 5A and 5B correspond to MoSC, in isolation (FIG. 5A) and gingivae MSC, in isolation (FIG. 5B). Morula stem cells are primitive benign stem cells. Gingivae MSC are mature mesenchymal stem cells from different tissues. The other mature mesenchymal stem cells can include: SHED (dental pulp, deciduous teeth); BMSC (Bone marrow stem cells); and UCMSC (Umbilical cord stem cells). Additionally, pulpal MSC were smaller in size than the morula stem cells (MoSC) isolated from gingivae.

### Example 6—Systems and Methods Herein Applied in Practice

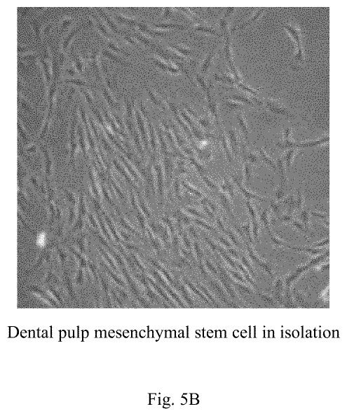

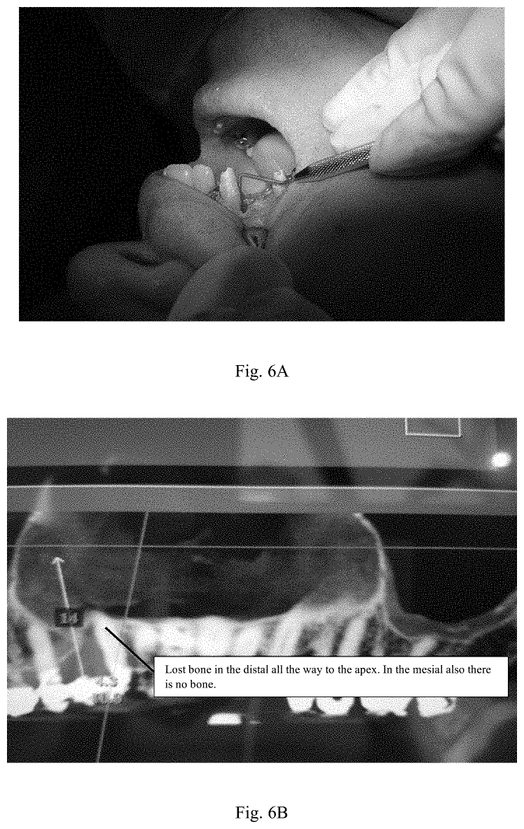

FIG. 6A is a situation of what dentists, periodontists and prosthodontists deal with on a daily basis, where there is a fenestration of the cortical bone at the apical area of canine #6 as a result of a chronic bacterial infestation. The dentist measured the pocket depth of the clinical destruction of the bone in the picture.

In FIG. 6B, a CT-scan shows bone loss in the mesial and distal aspect of canine #6. The area was disinfected with aPDT and in turn received PBM in preparation for bone grafting with morula stem cells and other biomaterials of the systems and methods herein. The point being made is that most periodontists are not able to grow bone back in this situation. The recommended treatment can be an extraction with either a bridge or a fixed (cemented) bridge of several units that require invasion of adjacent teeth, or an implant. With morula stem cells of the systems and methods herein, the patient was able to keep his teeth as the surrounding bone grew back. This is evidenced by comparing the before CT-scan with the post-treatment X-ray of the mature bone growth in the area that allowed canine #6 to support a fixed bridge.

### Example 7—Isolated Morula Stem Cells and Biomaterials Applicated

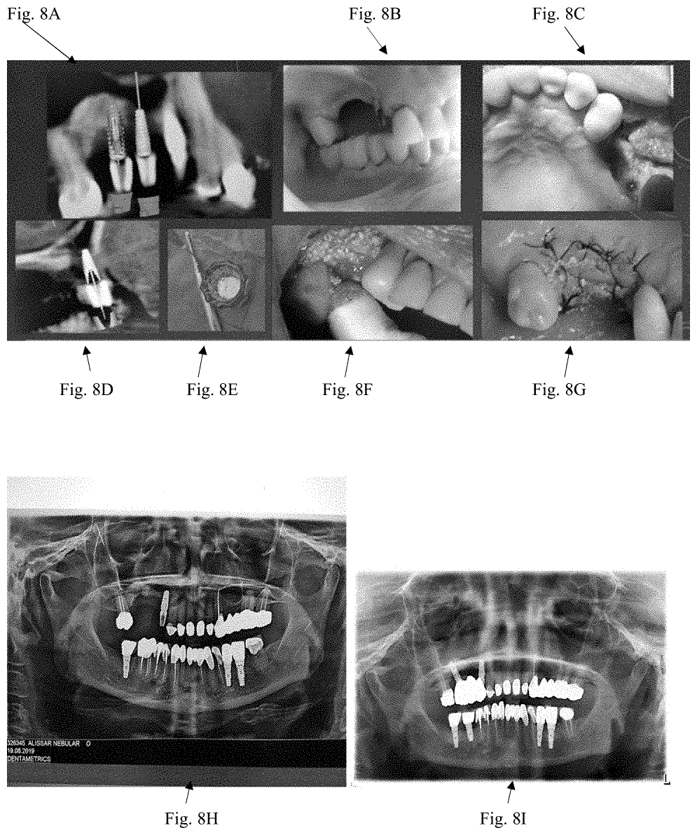

As depicted in FIGS. 8A-8I, the isolated morula stem cells proliferated and the paracrine MSC synergistically healed the site and produced enough bone to place an implant which later allowed an implant-supported bridge. More particularly, isolated MoSC were placed with bisulfate material [Bone Bond™ with Cerabone™ particles] and PRGF to produce a stimulating effect on the existing bone. Laser PBM in natural bone released calcitonnine and BMP2. The volume was placed in the first attempt leveled up at the neck of the neighboring tooth and covered all the threads of the neighboring implant. The vertical distance was 6 mm on the panoramic control x-ray. The width provided by the mix was 8 mm. The biological effect of isolated MoSC and the related biomaterials and macrophage polarization can be attributed to the distinct roles in the bone healing cascade, such as normal tissue repair encompassing a transition between a pro-inflammatory status to a pro-reparative status. When the first panoramic X-ray (FIG. 8H (before MoSC application)) is compared to the second (FIG. 8I (after MoSC application)), after surgery, there was an average of 6 mm growth of vertical bone in the upper right maxillae that is directly related to the application of isolated morula stem cells and Bone Bond™ with particles of bone and PRGF.

FIG. 8A: Digital diagnosis of two implants placed (int the area that corresponds to teeth numbered 3 & 4) in sagittal cut of bone defect at CT scan.

FIG. 8B: Crest of ridge with no bone width or height of bone enough for implant placement. Mesial implant 5 with uncovered distal surface, due to atrophied reabsorbed crest of ridge. Vestibular sagittal view.

FIG. 8C: Clinical condition of bone with oral antral communication due to bone defect distal to existing implant 5. Horizontal plane view.

FIG. 8D: Sagittal view of bone defect of oral antral communication with digital implant (4) placed at site.

FIG. 8E: Particles of bone and Bone Bond™ placed in sterile dapen dish.

FIG. 8F: Final contour (volume) of bone graft biomaterials (bone Bond™+particles of bone+PRGF) with Morula stem cells placed at crest of ridge.

FIG. 8G: Suture of the flat with Nylon.

FIG. 8H: Initial Panoramic x-ray showing non existing bone in the area correspondent to teeth 3 and teeth 4.

FIG. 8L Final panoramic x-ray, showing mature bone tissue at the previous defect site with a 3 Unit supported implant bridge. The distal implant resulted a positive support for the 3 Unit bridge.

### Example 8—Immunochemistry

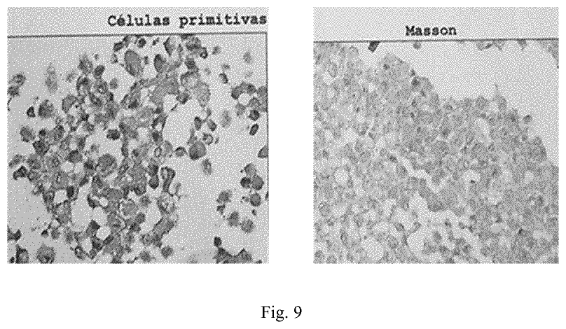

As depicted in FIG. 9, the samples showed primitive benign mesenchymal stem cells that are not differentiated, and their characteristics cannot be cataloged as fibroblasts. However, Masson tinting suggested this but is non-specific.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. A method for cell separation and growth comprising:
identifying and retrieving primitive benign morula autologous or allogenic and non-tumorgenic multi-potent cells as a biological growth platform, from gingiva tissues, wherein the biological growth platform creates cell lineages, tissues, organoids, transplanted organs, and organs;
using photobiomodulation on the biological growth platform, thereby stimulating separation of morula stem cells (MoSC) from the gingiva tissue;
responsive to stimulating the separation of the morula stem cells from the gingiva tissues, reproducing the morula stem cells in an environment in isolation from the gingiva tissues;
promoting stimulation of paracrine mesenchymal stem cells (MSC) and via addition of platelets rich in growth factors (PRGF) and the photobiomodulation (PBM), thereby generating tissues; and
adding biomaterials to the PRGF and MoSC to yield a biological mass at a junction of a patient, wherein the biological mass contains biological activity derived in part from the generated tissues.

2. The method of claim 1, wherein the biological mass at the junction of the patient is selected from the group consisting of: (1) bone tissue around teeth or dental implants; (2) rejuvenated vaginal wall; (3) urinary tract to retard incontinence; and (4) cell specific cell lineages for deriving organs.

3. The method of claim 1, wherein generating the tissues comprises using an allogenic ascendant to descendant transplantation in a direct line, thereby providing a structural platform within the patient to: repair arthritic/degenerative diseases, treat hearing loss, regrow lost hair, and treat mental diseases via exosomes.

4. The method of claim 1, wherein the biomaterials is osseo-conductive material selected from silicate phosphate cement, silicate lithium porcelain, and/or calcium phosphate (CaP) bioceramic material, dicalcium sulfate, titanium, zirconium, and/or other new materials for bone grafting in the human body.

5. The method of claim 1, wherein the morula stem cells are seeded or luted around margins within a matrix that preserves a microenvironment of molecules and proteins located at the imperceptible suture of the pre-maxillae/maxillae.

6. The method of claim 2, wherein the teeth is in a state associated with periodontal disease or a state associated with trauma from occlusion or other bone loss in the maxillae and/or mandible.

7. The method of claim 1, wherein the paracrine mesenchymal stem cells (MSC) maturing to the mature state construct, in part, the biological mass for treating ailments or illnesses in the patient.

8. The method of claim 7, wherein treating the ailments or illnesses in the patient comprises:
inhibiting autoimmune diseases due to the immunomodulatory capacity to enhance cell function, quantity, and quality of any cell type in identified autoimmune compromised patients;
mitigating cancer progression in identified cancer patients;
reversing symptoms and regenerate damaged tissues of SARS-Cov-2 and variants in identified Covid-19 patients;
attenuating and/or correcting mental diseases;
attenuating and/or correcting brain aneurysms;
attenuating hearing loss;
improving effects of aging in dermatological tissue and muscle tone;
improving erectile dysfunction;
inhibiting aging diseases of the eye composed of tear ducts, retina, iris, sclera and crystaline;
improving recovery from orthopedic injuries and orthopedic diseases;
inhibiting and curing diabetes;
inhibiting and/or repairing cardio-vascular conditions;
inhibiting and/or repairing diseases in hepatic systems;
inhibiting and/or repairing diseases in pulmonary systems;
inhibiting and/or repairing diseases in urinary systems;
inhibiting and/or repairing diseases respiratory systems;
inhibiting and/or repairing diseases in gastrointestinal systems;
inhibiting and/or repairing diseases in endocrine systems;
inhibiting and/or repairing diseases in lymphatic systems;
inhibiting and/or repairing diseases in reproductive systems;
inhibiting obesity;
transporting medical substances to targeted systems and organs; and
improving success rates of transplanted organs.

9. The method of claim 2, wherein the cell specific cell lineages for deriving the organs comprise the biological mass configured as a biologically active material for: creating new transplantable human organs; regrowing lost fingers; inhibiting of dental pulp tooth degeneration in the patient; growing new teeth in the patient; and inhibiting hair loss and supporting new cranial hair growth in the patient; growing periodontium in the patient; growing dental bone in the patient; growing muscle in the patient; growing tendons in the patient; growing neural tissue in the patient; regenerating dermatological tissues in the patient; and regenerating collagen in the patient.

10. The method of claim 9, wherein inhibiting of the dental pulp tooth degeneration in the patient comprises:
promoting the elimination of root canal treatment in the patient having necrotic pulp and apical fenestration on the cortical of the maxillae/mandible;
promoting elimination of inflammation in the patient with inflammatory process of the dental pulp due to decay wherein the dental pulp is vital; and
promoting sealed dentinal tubules wherein the pulp is vital via photobiomodulation.

11. The method of claim 9, wherein growing the new teeth in the patient comprises:
promoting the new tooth growth in the patient who has lost a tooth in the maxillae or mandible having a dormant dental follicle generating a supernumerary tooth in-vivo;
creating a mix of bone bond and particles of bone with the morula stem cells in the proportion of 0.2-0.8 cubic centimeter (cc) of bone bond and 0.2-0.8 cc particles of bone at 50/50 proportion with 10-15 drops of the morula stem cells to create a thick paste molded with a hand dental instrument during surgery into the existing defect within the junction of the patient and sutured shut with nylon 3-0 caliber stitches, thereby stitches remain in place for 12-20 days and are configured to be removed by a dentist; and
injecting the morula stem cells into a maxillae or mandible area associated with tooth loss, wherein exosomes of adipose tissue extraction are administered to the treatment area via inhalation method for approximately 15-50 minutes and photobiomodulation at a 905 nm or 976 nm wavelength at 0.6-1.0 Joules/square centimeter administered to the treatment area after inhalation within 1-2 hours, wherein the exosomes are allogenic or autologous.

12. The method of claim 9, wherein inhibiting the hair loss and supporting the new cranial hair growth comprises:
injecting 2 drops of saline solution containing approximately 4,000-5,000 morula stem cells into the cranial dermis where hair loss is observed; and
using a syringe type device to perforate derma at 0.25-0.50 mm depth, wherein the syringe type device contains a saline solution with morula stem cells in an allogenic and/or autogenic state, thereby administering: (1) multiple injections of the saline solution and (2) exosomes of adipose tissue extraction to a treatment area via an inhalation method for approximately 15-50 minutes and photobiomodulation at 905 nm or 976 nm wavelength at 0.6-1.0 Joules/square centimeter administered to the treatment area after the inhalation within 1-2 hours, wherein the exosomes are allogenic or autologous.

13. A biological mass at a junction of a patient comprising:
paracrine mesenchymal stem cells (MSC) stimulated to a mature state from morula stem cells, via addition of platelets rich in growth factors (PRGF) and photobiomodulation; and
biomaterials added to the PRGF such that the MSC form a biological mass at a junction of a patient,
wherein the biomaterials is osseo-conductive material selected from silicate phosphate cement, silicate lithium porcelain, and/or calcium phosphate (CaP) bioceramic material, dicalcium sulfate, titanium, zirconium, and/or other new materials for bone grafting in the human body.

14. The biological mass of claim 13, further comprises exosomes of MoSC or adipose tissue extraction, wherein the exosomes are allogenic or autologous.

15. A consumer or medical product comprising:
isolated morula stem cells (MoSC),
and/or exosomes, and
excipient ingredients,
wherein the consumer or medical products are: pharmaceutical products, medical and veterinary preparations, hygiene and sanitary products for medical use, foods and dietary substances of medical and veterinary use, baby food, food supplements for humans and mammals, dressing materials and poultice, dental impression materials and dental liners, or dental cements, disinfectants, products to eliminate harming animals, and fungicides.
